(19) United States
(12) Patent Application Publication
(10) Pub. No.: US 2008/0260868 A1
(43) Pub. Date: Oct. 23, 2008

(54) Folate based composition for neurological and cognitive applications

(76) Inventor: Curt Hendrix, Encino, CA (US)

Correspondence Address: VENABLE LLP, P.O. BOX 34385, WASHINGTON, DC, 20043-9998, US

(21) Appl. No.: 12/149,075

(22) Filed: Apr. 25, 2008

Related U.S. Application Data
(63) Continuation of application No. 11/293,425, filed on Dec. 1, 2005, now abandoned.
Application No. 11/293,425 is a continuation-in-part of application No. 11/116,997, filed on Apr. 27, 2005, now abandoned.
Application No. 11/116,997 is a continuation-in-part of application No. 11/002,750, filed on Dec. 1, 2004, now abandoned.
(60) Provisional application No. 60/632,681, filed on Dec. 1, 2004.
(60) Provisional application No. 60/632,681, filed on Dec. 1, 2004.

(51) Int. Cl. A61K 36/82; A61K 31/225; A61K 31/385; A61K 47/12; A61K 31/355; A61K 31/353; A61K 31/44; A61K 31/714; A61K 31/445; A61K 31/195; A61P 9/10

(52) U.S. Cl. 424/729; 514/547; 514/440; 514/784; 514/458; 514/456; 514/345; 514/52; 514/321; 514/563

(57) ABSTRACT

New formulations for the prevention and treatment of neurological diseases and cognitive deficiencies, particularly Alzheimer's Disease comprise folate in combination with compounds chosen to address some or all of the pathways which can result in neurological deficiencies and diseases, namely inflammation, oxidative stress, glycation/dysinsulinemia, platelet function, homocysteine levels and acetylcholinesterase inhibition, that are important contributors to the development or progression of AD.

This application is a Continuation-in-Part of Ser. No. 11/002,750 filed Dec. 1, 2004 and Ser. No. 11/116,997 filed Apr. 27, 2005 and claims benefit of Provisional Application No. 60/632,681 filed Dec. 1, 2004.

This application is directed to new formulations for the prevention and treatment of neurological diseases and cognitive deficiencies, i.e., Alzheimer's Disease (AD), Parkinson's Disease, ALS and other types of dimentia which comprise folate in combination with compounds chosen to address some or all of the pathways which can result in neurological deficiencies, degeneration and diseases.

### BACKGROUND

Folic acid or salts thereof, referred to as folates, along with vitamins B₆ and B₁₂ are required in metabolic pathways involving methionine, homocysteine, cystathionine, and cysteine. The term folates as used herein is meant to include, as a minimum, folacin (USP folic acid), naturally occurring folinic acid, 5-methyl tetrahydrofolate, and tetra hydrofolate as well as salts or metabolites of these compounds. It appears that all three compounds (Folate, B₆ and B₁₂) are necessary for normal metabolism. However, these three compounds each function in a different manner. Folate, even if available at normal levels, is consumed in the metabolic process and therefore must be constantly replenished by diet or supplements. However, B6 and B12 function as co-factors. While necessary for the metabolic process to proceed, they are each regenerated in the process. Therefore, if they are present in normal amounts in serum, supplementation may not be necessary. B₁₂ in the form of 5′-deoxyadenosylcobalamin is an essential cofactor in the enzymatic conversion of methylmalonylCoA to succinylCoA. The remethylation of homocysteine (HC) to methionine catalyzed by methionine synthase requires folate (methyltetrahydrofolate) and B₁₂ in the form of methylcobalamin. HC is condensed with serine to form cystathionine (CT) in a reaction catalyzed by cystathionine beta.-synthase which requires B₆ (pyridoxal phosphate). CT is also hydrolyzed in another B₆-dependent reaction to cysteine and alpha.-ketobutyrate. Homocysteine is a modified form of the amino acid methionine that is tightly regulated by enzymes which require folate. By impairing DNA repair mechanisms and inducing oxidative stress, homocysteine can cause the dysfunction or death of cells in the cardiovascular and nervous systems. Homocysteine appears to be present in many disease states. However, dietary folate stimulates homocysteine removal and may thereby protect cells against disease processes.

The principal biochemical function of folates is the mediation of one-carbon transfer reactions. 5-Methyltetrahydrofolate donates a methyl group to homocysteine, in conversion of homocysteine to L-methionine. The enzyme that catalyzes the reaction is methionine synthase. Vitamin B₁₂ is a cofactor in the reaction. This reaction, in which folate and vitamin B₁₂ are coparticipants, is of great importance in the regulation of serum homocysteine levels. The L-methionine produced in the reaction can participate in protein synthesis and is also a major source for the synthesis of S-adenosyl-L-methionine (SAMe). The methyl group donated by 5-methyltetrahydrofolate to homocysteine in the formation of L-methionine is used by SAMe in a number of transmethylation reactions involving nucleic acids, phospholipids and proteins, as well as for the synthesis of epinephrine, melatonin, creatine and other molecules. Tetrahydrofolate is the folate product of the methionine synthase reaction. 5-Methyltetrahydrofolate is generated by conversion of 5,10-methylenetetrahydrofolate into 5-methyltetrahydrofolate via the enzyme methyleneterahydrofolate reductase (MTHFR). 5,10-Methylenetetrahydrofolate is regenerated from tetrahydrofolate via the enzyme serine hydroxymethyltransferase, a reaction, which in addition to producing 5,10-methylenetetrahydrofolate, yields glycine.

5,10-Methylenetetrahydrofolate, in addition to its role in the metabolism of homocysteine, supplies the one-carbon group for the methylation of deoxyuridylic acid to form the DNA precursor thymidylic acid. This reaction is catalyzed by thymidylate synthase and the folate product of the reaction is dihydrofolate. Dihydrofolate is converted to tetrahydrofolate via the enzyme dihydrofolate reductase.

Folates are also involved in reactions leading to de novo purine nucleotide synthesis, interconversion of serine and glycine, generation and utilization of formate, the metabolism of L-histidine to L-glutamic acid, the metabolism of dimethylglycine to sarcosine and the metabolism of sarcosine to glycine.

One of the natural folates, folinic acid, is used as a pharmaceutical agent. Folinic acid, also known as leucovorin, citrovorum factor and 5-formyltetrahydrofolate, is used as rescue therapy following high-dose methotrexate in the treatment of osteosarcoma. It is also used to diminish the toxicity of methotrexate. It is used in the treatment of megaloblastic anemia due to folate deficiency and in the prevention or treatment of the toxic side effects of trimetrexate and pyrimethamine. The combination of folinic acid and 5-fluorouracil has until recently been standard therapy for metastatic colorectal cancer. Folinic acid increases the affinity of fluorouracil for thymidylate synthase. Folinic acid is available as a calcium salt for parenteral or oral administration.

In addition to being known as pteroylglutamic acid or PGA, folic acid is known chemically as N-[4-[[(2-amino-1,4-di-hydro-4-oxo-6-pteridinyl)methyl)amino]benzoyl]-L-glutamic acid. Older names for folic acid are vitamin B₉, folicin, vitamin Bc and vitamin M. Its molecular formula is C₁₉H₁₉N₇O₆ and its molecular weight is 441.40 daltons. Folic acid forms yellowish-orange crystals. The color is imparted by the pteridine ring of folic acid. Pteridine also imparts color to butterfly wings.

Folate has been prescribed as a nutritional supplement for many medical conditions based on the presence of elevated homocysteine levels which occur in those conditions. Folate supplements appear to reverse the elevated homocysteine levels. However, the elevated homocysteine level may be a result of inadequate supply or excessive consumption of folate and not the cause of the disease. It is clinically beneficial in such instances to provide folate supplements as individuals with elevated homocysteine levels appear to be at increased risk for cardiovascular disease and stroke, and neurodegenerative disorders such as Alzheimer's and Parkinson's diseases as well as neural tube defects, spontaneous abortion, placental abruption, low birth weight, renal failure, rheumatoid arthritis, alcoholism, osteoporosis, neuropsychiatric disorders, non-insulin-dependent diabetes and complications of diabetes, fibromyalgia and chronic fatigue syndrome. Moderate elevations of HC might be associated with increased risk for vascular disease (Ueland et al. (1992) in Atherosclerotic Cardiovascular Disease, Hemostasis, and Endothelial Function (Francis, Jr., ed.), Marcel Dekker, Inc., New York, pp. 183-236). However, folic acid deficiencies have also been associated with peripheral vascular disease and coronary disease in individuals with normal homocysteine levels (Bunout, D. et al “Low Serum Folate but Normal Homocysteine Levels in Patients with Atheroslerotic Vascular Disease and Matched Healthy Controls”, Nutrition 2000, 16, p434-8) suggesting that folates may have a protective effect that extends beyond maintaining normal homocysteine levels. In addition, moderate hyperhomocysteinaemia has been shown to be frequently present in cases of stroke and to be independent of other stroke risk factors (Brattstrom et al. (1992) Eur. J. Clin. Invest. 22:214-221).

It is not clear if the various disease states are caused by elevated homocysteine levels or the elevated homocysteine levels are caused by other factors which are the primary cause of the disease state and result in elevated levels of homocysteine. For example, it is also known that folate supplements are usefully where B₁₂ deficiencies exist, but homocysteine levels may not be elevated. Individuals with B₁₂ deficiency can display neurologic disorders, typically relating to underlying anemia. However, supplementing diet with only folate is not medically recommend as these folate supplements may mask the underlying B₁₂ problem. U.S. Pat. No. 4,945,083, issued Jul. 31, 1990 to Jansen, entitled Safe Oral Folic Acid-Containing Vitamin Preparation, describes an oral vitamin preparation comprising the combination of 0.1-1.0 mg B₁₂ and 0.1-1.0 mg folate for the treatment or prevention of megaloblastic anemia.

Normal serum folate levels in healthy individuals are 2.5-20 ng/ml, with levels less than 2.5 ng/ml indicating the possibility of clinically significant deficiency. Like B₁₂ serum levels, however, serum folate levels are a relatively insensitive measure in that only 50-75% of patients with folate deficiency have levels less than 2.5% ng/ml, with most of the remaining 25-50% being in the 2.5-5.0 ng/ml range (Allen (1991), Cecil Textbook of Medicine, 19th Ed.).

A series of patents to Allen et al, (U.S. Pat. No. 5,563,126, U.S. Pat. No. 5,795,873, U.S. Pat. No. 6,207,651, U.S. Pat. No. 6,297,224 and U.S. Pat. No. 6,528,496)) teaches the use of oral compositions or a transdermal patch delivering a combination of B₁₂ and folate, or B₁₂, folate and B6, in concentrations sufficient to reduce elevated homocysteine levels by treating either single or multiple deficiencies of B₁₂, folate, and B₆. The Allen non-prescription formulations include 0.3-10 mg CN-cobalamin (B₁₂) and 0.1-0.4 mg folate or 0.3-10 mg B₁₂, 0.1-0.4 folate, and 5-75 mg B₆. The Allen prescription formulations comprise between 0.3-10 mg CN-cobalamin (B₁₂) and 0.4-10.0 mg folate or 0.3-10 mg B₁₂, 0.4-1.0 mg folate, and 5-75 mg B₆.

The standard of care for patients with Alzheimer's Disease is treatment with anticholinesterase inhibitors, currently the only approved treatment. Cholinesterase inhibitors increase the synaptic availability of the neurotransmitter acetylcholine by preventing it from breaking down. Anticholinesterase inhibitors act to stabilize progression of the disease (particularly cognitive function and overall functioning) and often delay the need for institutionalization by several months. Unfortunately, the effect of cholinesterase inhibitors is only temporary. No treatment currently exists that prevents, halts, or reverses the neurodegenerative process.

### SUMMARY

New formulations for the prevention and treatment of neurological diseases and cognitive deficiencies and particularly Alzheimer's Disease (AD), Parkinson's Disease, ALS and other types of dimentia comprise folate in combination with compounds chosen to address some or all of the factors, pathways or mechanisms that relate to oxidative stress, glycosylation, inflammation and platelet function which can result in neurological deficiencies, degeneration and diseases.

### BRIEF DESCRIPTION OF THE DRAWINGS

FIG. 1 is a schematic drawing of the pathophysiological processes involved in Alzheimer's disease.

### DESCRIPTION OF INVENTION

Set forth herein is a medical food cocktail that can slow, halt or reverse the development of Alzheimer's Disease during the early stages of the disease. The cocktail is composed of nutritional ingredients that are demonstrated in the basic science and clinical medical literature to impact those specific biochemical and physiological processes thought to contribute to the onset and development of Alzheimer's Disease. These ingredients are all currently listed as Generally Accepted As Safe (GRAS) by the FDA, or are self-affirmed as GRAS ingredients, or in common use as dietary supplements. In addition, applicant has discovered that dietary supplementation with folate may be beneficial in treating certain medical conditions. In particular, compositions set forth herein, which include folates, have been found to be beneficial in preventing, reducing the severity of, or reversing various neurological diseases or cognitive disorders, including but not limited to Alzheimer's Disease, even though the individual does not appear to have a B₁₂ deficiency or elevated homocysteine levels. These compositions may also be beneficial in preventing B₁₂ deficiencies or elevated homocysteine levels.

An objective of the invention is to provide a formulation for a medical food cocktail to be used for the prevention and treatment of Alzheimer's Disease. The cocktail will consist of standardized herbal extracts, vitamins and vitamin metabolites, and minerals that are currently listed by the FDA as generally recognized as safe (GRAS) or are self-affirmed as GRAS ingredients or are commonly used in dietary supplement. Included are ingredients that have been shown in the basic science and clinical medical literature to affect cognitive function and/or biochemical or pathophysiological processes known to be involved in Alzheimer's Disease

S-adenosylmethionine (SAMe) is a substance that occurs naturally in the body. It is the combination of one (1) essential amino acid and ATP that plays a role in 35-40 biochemical reactions throughout the body. In most people, the body can make all the SAMe it needs, but some patients with depression and other psychological conditions have been found to have lower levels of the compound as well as lower levels of folate and vitamin B₁₂. These three substances each play a part in the metabolic process of “methyl donation” or “methylation”, a process in which a molecule comprised of one (1) carbon molecule and three (3) hydrogen atoms is attached to proteins and lipids. These methylation reactions are involved in the production of the neurotransmitters serotonin and dopamine in the brain and enzymes that help repair joints and the liver. There is evidence that serotonin is a factor in migraine and is involved in the so called “rebound effect”, because of its vasoconstricting effect when serotonin levels are elevated and subsequent vasodilation as serotonin levels decrease. Coincidentally, folate deficiency also appears to reduce brain serotonin and contribute to depression in individuals. By supplementing the diet with folate, serotonin generation and its metabolism is balanced, depression decreases and the cycling of vasodilation and vasoconstriction caused by fluctuation in serotonin is minimized.

Based on a review of the literature on Alzheimer's Disease several markers and/or chemical processes have been identified that either contribute to the development of neurological or cognitive deficiencies, particularly AD, or are present in higher amounts in individuals diagnosed with AD. These are referred to herein as AD Factors. However, these factors are not limited to Alzheimer's and are found in various neurological and cognitive deficiencies. Several active compounds are identified which can be used to address these AD Factors. Polytherapy, namely the use of a cocktail or mixture of these active compounds to prevent, slow or reverse Alzheimer's Disease, Parkinsons, ALS and other types of dimentia, are set forth herein to address the multiple factors associated with the etiology or progression of the disease. Applicant has now combined several of those ingredients to reduce the dementia caused by AD, or decrease or prevent the markers or biochemical events and to be beneficial in preventing, slowing or reversing the effects of AD in human subjects.

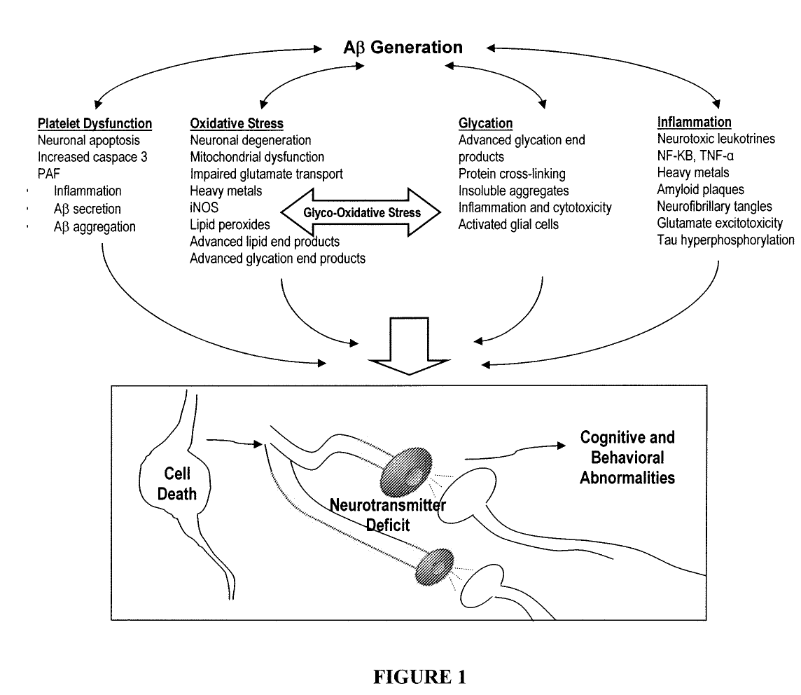

Applicant has addressed the 4 major biochemical phenomena or pathways, namely inflammation, oxidative stress, glycation/dysinsulinemia, and platelet function set forth in FIG. 1, and a key marker, homocysteine levels, that are important contributors to the development or progression of AD.

An additional factor, acetylcholinesterase inhibition, is addressed by currently existing drugs (Aricept™ (donepezil), Exelon™ (rivastigmine) and Reminyl™ (galantamine). Additionally, Namenda™ (memantine) is used to prevent toxic levels of glutamate, also a chemical messenger, in the brain. Four pathways and their associated mechanism, markers and factors which differ in some respects from FIG. 1 by the addition of some additional factors, are set forth in Table 1. Several naturally occurring compounds or group of compounds have been identified by applicant to decrease, reverse or prevent these phenomena from occurring. While use of each separately is beneficial in treating AD, applicant has discovered that there is a synergistic benefit in combining three or more of these compounds into a cocktail. Each compound addresses one or more of the 4 different mechanisms or pathways which contribute to AD as well as other neurological deficiencies and diseases. Further, the combination creates an environment where it is difficult for beta-amyloid plaques to either develop or deposit.

TABLE 1
A.D. Associated Mechanisms, Markers and Factors Associated
with or Impacted by the 4 Biochemical Categories

| | | | Platelet |
|---|---|---|---|
| Oxidative Stress | Glycosylation | Inflammation | Function |
| Mitochondrial Dysf. | MMP prod. | Tau and | Secretion of |
| Glutamate Transport | Oxidation | β Amyloid | Aβ-Glutamate |
| Beta Amyloid | Inflammation | MMP Excit. | Inflammation |
| iNos | β-Amyloid | Homocysteine | PAF |
| Heavy Metals | toxicity | Heavy Metals | Aggregation + |
| Ubiquitin-Proteos. | Cognition | Mitochon. Dysf. | Capsase 3 |
| Heat Shock Proteins | Mitochon. Dysf | TNF-β, NF-KB | |
| Platelet Act. Factor | Adv. Glyc. | Plat. Act. Fact. | |
| Cognition | Endprod. | | |
| TBARS, | Pentosidine, | | |
| Malondialdehye | CML | | |
| 4-Hydroxnonenal | | | |

While a single cause for Alzheimer's Disease has not been identified, the brain of people diagnosed with AD typically exhibit sticky plaque composed of amyloid protein deposits as well as tau protein tangles. The prevention of plaque and tangles, or its reversal/reduction if formed, is addressed by the invention.

Inflammation—Chronic inflammation damages host tissue, brain neurons are particularly vulnerable. Inflammatory mediators are produced and elevated in affected regions of brains of individuals with AD. Non-Immune mediated chronic inflammatory responses in brain parenchyma in response to beta Amyloid (Abeta) peptides are believed to be involved in AD progression. Neurodegenerative plaques of AD are characterized by an up-regulation of interleukin-1 and interleukin-6 and this up-regulation can play a role in the pathogenesis of AD. Advanced glycation end products have been shown to exert an inflammatory effect as well. The invention described herein uses the therapeutic benefits of naturally occurring compounds to slow or halt the chronic inflammatory-like process that occurs in the early pathological cascade of AD. Markers of inflammatory response include serum alpha (1) anti-chymotripsin, NF-kappaBeta, high sensitivity C-reactive protein, platelet activation factor, transforming growth factor beta, TNF-alpha and inflammatory cytokine production in general. An inflammatory cascade precipitated by the formation of Abeta plaques in the brain is thought to be a prime cause of neuronal death. The inflammatory marker C-reactive protein and microglial inflammatory markers are all upregulated in tissue from Alzheimer's patients. C-reactive protein-like inflammation has been demonstrated in both the senile protein plaques and neurofibrillary tangles of Alzheimer's victims Chronic inflammation may also be responsible for the degeneration of the hippocampus, a particularly vulnerable part of the brain. Naturally occurring compounds (phytochemicals) that have a beneficial impact on inflammation can be beneficial in prevention of AD and in slowing its progression, especially because many of these processes are measurable long before clinical symptoms appear.

Oxidative Stress—Like Inflammation, oxidative stress plays a role in the development and progression of most chronic degenerative diseases of which AD is no exception. Alzheimer diseased brains are characterized by excessive Abeta deposition and by extensive oxidative stress. Sources of oxidative stress are multiple and include advanced glycation end products, microglial activation and the sequelae of Abeta. Membrane permeable antioxidants prevent the up-regulation of induced nitric oxide synthase (iNOS) and can be viewed both as antioxidants as well as anti-inflammatory drugs. The destructive free radicals produced by oxidative stress damage sensitive neurons. Metals such as iron, copper, zinc, and aluminum exacerbate the production of free radicals, as does the presence of Abeta plaques, creating a vicious cycle of neuronal damage. Alzheimer's patients also exhibit high serum levels of markers of oxidative stress and low plasma levels of antioxidants and free radical scavengers. Evidence from the medical literature suggests that nutritional antioxidants can block or reduce neuronal death. Treatment with antioxidants is also beneficial in preventing and/or slowing AD. Of particular interest are combinations of antioxidants that have complementary or synergistic activity or quench multiple types of reactive oxygen species.

Glycation/Dysinsulinemia—Glycation, the reaction of proteins with sugars to produce advanced glycation end-products (AGEs), is a major cause of the physical manifestations of aging and damage to tissue elasticity. Extracellular AGEs accumulate in the Abeta plaques of Alzheimer's patients, causes further oxidative stress on the surrounding neural tissue. AGEs are also found in the serum and cerebral spinal fluid of Alzheimer's patients An increasing percentage of adults and children are overweight; obesity often causes dysinsulinemia that can lead to increased glycation of proteins. There is an increasing tendency toward Non-Insulin Dependent Diabetes Mellitus (NIDDM) even in people within normal body mass indicies (BMIs). This trend, coupled with the potential effects of glycation on all types dementia is of concern. Glycoxidative (glycation+oxidation) stress creates a cascade of events leading to neurodegeneration in AD. The accumulation of advanced glycation end products (AGEs) explain neuro-pathological and biochemical events such as protein cross linking, free radical damage, neuronal apoptosis and glial activation that are features of AD. Several markers of glycoxidative stress have been identified. Examples of these markers are pentosidine, N(epsilon)-(carboxymethyl)lysine(CML), fructosamine, malondialdehyde(MDA), 4-hydroxy-2-noneal (HNE) which can be quantitatively measured in patients. Several naturally occurring ingredients (AGE Inhibitors), discussed below, can slow, halt or reverse glycoxidative effects on AD.

Platelet Function—Platelets are a source of beta-amyloid precursor protein. Increased platelet activation, abnormal platelet function and increased circulating beta-amyloid has been identified in AD. Activated platelets are a source of Abeta peptides and Beta-amyloid aggregates platelets and supports their adhesion. There is considerable in vitro evidence that non-steroidal anti-inflammatory drugs (NSAIDs) can reduce the inflammatory response of microglial cells. Ingredients that are both anti-inflammatory and normalize platelet function are beneficial as therapeutic options in AD. A significant correlation exists between platelet activating factor (PAF) binding and degree of cognitive impairment in Alzheimer's patients. Similarly, neurons pretreated with PAF antagonists rendered the neurons resistant to damage by Abeta and also reduced activation of caspase-3, a marker of apoptosis (programmed cell death) Ingredients meeting both of these requirements, discussed below in regard to the therapeutic cocktail, have been found to be more effective when combined with other active compounds than single agents for AD.

Homocysteine—Homocysteine, discussed above, is believed to be a marker of, or a risk factor for, both stoke and cardiovascular disease. It has been estimated that exceeding normal levels (5-15 micromol/L) by as little as 5 micromol/L increases the risk of coronary artery disease by 60 percent in men and 80 percent in women. Researchers at the Boston University School of Medicine have also provided convincing evidence that high homocysteine levels are also a risk factor for Alzheimer's disease. Their study involved 1092 men and women, between 1986 and 1990, with an average age of 76 years who were deemed to be free of dementia when examined as part of the Framingham Study. Eight years later 111 of the study participants had developed dementia and 83 of them were diagnosed as having Alzheimer's disease. The researchers found that individuals with a blood plasma homocysteine level above 14 micromol/L had nearly twice the risk of developing Alzheimer's disease as did people with lower levels. They also determined that a 5 micromol/L increase in homocysteine level corresponds to a 40 percent increased risk of Alzheimer's disease. However, it is not clear if these effects are the result of high homocysteine levels or of a folate deficiency which also result in elevated homocysteine, i.e., a marker of a disease condition. It has been discovered in studies on mice that folate deficient diets can result in nerve damage. Also this damage can be halted and even reversed by repair of nerve cell DNA damage in the brain. The Alzheimer's Cocktail.

A preferred composition for use in preventing, treating or reducing the severity of AD comprises a combination of three or more of curcumin, alpha lipoic acid, N-acetylcysteine, Vitamins C and E, epigallocatechin-3-gallate (from green tea extract) and B-complex (B-1, B-5, B-6, B-12 and folate), L-carnosine, protolytic enzymes and piperin. Table 2 lists the AD Factors and the compounds proposed to address each. It should be noted as discussed below, several of these compounds address more then one of these factors. In addition, several of the ingredients have been shown to exhibit antocholinesterase activity. Further, there are no known maximum daily dosage levels for these compounds or they are not toxic unless consumed in very high quantities and they are generally recognized to be safe for daily consumption.

Curcumin is a polyphenol that comprises the active component of the plant/spice referred to as turmeric (Curcuma longa). The root and rhizome of turmeric have been used medicinally. The plant extract is standardized to 90-95% curcumin or curcuminoids.

TABLE 2
Medical Food Cocktail Ingredients
and Disease Processes Targeted

| | Effect on Biochemical Process? | | | |
|---|---|---|---|---|
| | Oxidative | | | Platelet |
| Cocktail Ingredient | Stress | Inflammation | Glycation | Function |
| Curcurmin | * | * | * | * |
| Piperine | * | * | * | * |
| Epigallocatechin-3- | * | * | | * |
| gallate (EGCG) | | | | |
| α-Lipoic Acid | * | * | | * |
| N-Acetylcysteine | * | * | * | |

B Vitamins

| | | | | |
|---|---|---|---|---|
| B1 | | | * | |
| B6 | * | * | * | * |
| B12 | * | * | | |
| Folate | * | | | |
| Vitamin C | * | * | | |
| Vitamin E | * | * | | * |

It is a strong antioxidant, is a potent inhibitor of lipid peroxidation and has several anti-inflammatory mechanisms including the lowering of histamine levels and the potential of increasing natural cortisone production by the adrenals and modulating specific interleukins, cytokines, leukotrienes and eicosanoid synthesis in general. Curcumin has been shown to modulate many inflammatory markers such as TNF-a and NF-Kappa-b. It also provides hepatoprotective benefits against a number of toxic compounds. Recent studies indicate that curcumin also demonstrates anti-platelet effects which may protect against beta amyloid induced platelet aggregation and platelet adhesions, has shown anti-glycation benefits and has been shown to decrease platelet-activating factor (PAF) which disrupts normal platelet function. Curcumin was found to protect normal human umbilical vein endothelial cells from beta amyloid (Abeta). In studies on mice, low dose curcumin significantly lowered oxidized proteins and IL-1beta in mice brains and suppresses Abeta induced cognitive defects and oxidative damage. Using low dose curcumin, insoluble beta-amyloid, (Abeta), soluble Abeta, and plaque burden were decreased by 43-50%.

In Alzheimer transgenic mice, dietary curcurmin was associated with decreased levels of oxidized proteins and interleukin-1 beta (a marker of inflammation), as well as a 43-50% decrease in insoluble Abeta, soluble Abeta, and amyloid plaque burden. A suppression of microgliosis in both studies has also been observed. In addition, curcurmin has been shown to prevent the accumulation of advanced glycation endproducts in diabetic rats receiving dietary curcumin (200 mg/kg body weight) compared to control diabetic rats without curcurmin. The same study showed a significant reduction in lipid peroxidation products (indicators of oxidative stress) in the curcumin fed rats. The investigators noted that the preventative effects of curcurmin were more pronounced than the therapeutic effects.

Data from in vitro studies also provide evidence that curcurmin may be beneficial in the prevention and treatment of Alzheimer's disease. Curcurmin has been shown to inhibit both the formation and growth of beta-amyloid fibrils from Abeta in a dose-dependent manner. In in vitro studies on rat glioma and mixed neuroglial cells, curcurmin inhibited neuroglial proliferation. In a neuroblastoma cell line, curcumin inhibited activation of the inflammatory marker NKFB (41). Likewise, curcumin inhibited inflammation-related cyclooxygenase-2 gene expression in microglial cells. Curcurmin also inhibits platelet activating factor (PAF) and platelet aggregation induced by platelet agonists. Other studies in animal models of Alzheimer's suggest that curcumin acts as metal chelator, thus reducing Abeta aggregation and toxicity, while suppressing damage from inflammation. Along this line, curcurmin has been shown to chelate both cadmium and lead in rat brain homogenates, protecting against lipid peroxidation. Supplementation with tumeric reduces oxidative stress and attenuates the development of fatty streaks in rabbits fed a high cholesterol diet. Curcumin is preferably present in daily dosages of at least about 2250 mg.

Alpha Lipoic Acid (ALA), a disulfide molecule (a compound containing two thiol groups), is a unique antioxidant that is both lipid and water soluble and promotes synthesis of the endogenous antioxidant, glutathione. Studies indicate that ALA enhances glucose uptake, inhibits glycosylation and improves peripheral neuropathies and associated nerve pain. ALA has demonstrated the ability to prevent AGE induced increases in NF-kappa-b activation, thus protecting against endothelial dysfunction. In a small open label study ALA was shown to stabilized cognitive function in elderly, beginning stage Alzheimer patients. We have investigated the potential effectiveness of alpha-lipoic acid (ALA) against cytotoxicity induced by Abeta peptide (30 microM) and hydrogen peroxide (H₂O₂) (100 microM) with the cellular 3-(4,5-dimethylthiazol-2-yl)-2,5-diphenyl-tetrazolium bromide (MTT) reduction and fluorescence dye propidium iodide assays in primary neurons of rat cerebral cortex. It was found that treatment with ALA protected cortical neurons against cytotoxicity induced by Abeta or H₂O₂.

It was further found that AGEs induce lipid peroxidation in a neuronal cell line in a dose-dependant manner, and that blocking the specific AGE-receptor RAGE, as well as using different antioxidants (alpha-lipoic acid, N-acetylcysteine, 17 beta-estradiol or aminoguanidine) can reduce the AGE-mediated formation of lipid peroxidation products. Extracellularly administered alpha-lipoic acid reduces AGE-albumin-induced endothelial expression of VCAM-1 and monocyte binding to endothelium in vitro as well as demonstrating significant antioxidant potential. ALA is preferably present in daily dosages of at least about 50 mg.

N-Acetylcysteine (NAC) was studied versus a placebo administered in a double-blind fashion to patients who met National Institute of Neurological and Communicative Disorders and Stroke-Alzheimer's Disease and Related Disorders Association criteria for probable AD. Testing for efficacy occurred after 3 and 6 months of treatment. Comparison of interval change favored NAC treatment on nearly every outcome measure, although significant differences were obtained only for a subset of cognitive tasks. Oxidative stress may play a crucial role in age-related neurodegenerative disorders. The ability of two antioxidants, alpha-lipoic acid (ALA) and N-acetylcysteine (NAC), to reverse the cognitive deficits found in the SAMP8 mouse has been examined. By 12 months of age, this strain develops elevated levels of Abeta and severe deficits in learning and memory. 12-month-old SAMP8 mice, in comparison with 4-month-old mice, had increased levels of protein carbonyls (an index of protein oxidation), increased TBARS (an index of lipid peroxidation) and a decrease in the weakly immobilized/strongly immobilized (W/S) ratio of the protein-specific spin label MAL-6 (an index of oxidation-induced conformational changes in synaptosomal membrane proteins). Chronic administration of either ALA or NAC improved cognition of 12-month-old SAMP8 mice in both the T-maze footshock avoidance paradigm and the lever press appetitive task without inducing non-specific effects on motor activity, motivation to avoid shock, or body weight. These effects are believed to have occurred directly within the brain, as NAC crossed the blood-brain barrier and accumulated in the brain. Furthermore, treatment of 12-month-old SAMP8 mice with ALA reversed all three indexes of oxidative stress. These results support the hypothesis that oxidative stress can lead to cognitive dysfunction and provide evidence for a therapeutic role for antioxidants. NAC has also been shown to antagonize N-methyl-D-aspartate (NMDA) caused glutamergic excitation and its neurotoxicity. NAC is preferably present in daily dosages of at least about 100 mg.

Vitamins C and E are well known for their anti-oxidant properties. In a study of more than 4740 subjects in Cache County, Utah, use of vitamin C and E supplements was associated with a significant reduction in risk of Alzheimer's disease. Similar results were seen in the Honolulu-Asia aging study of 3385 elderly men: vitamin C and E supplement were associated with a protective effect for vascular and mixed dementia. Dementia patients and Alzheimer's patients also exhibit lower plasma vitamin C concentrations than control subjects with no cognitive impairment. Vitamin E has been shown to prevent increased protein oxidation, reactive oxygen species, and Abeta-induced neurotoxicity in a rat embryonic hippocampal neuronal culture. Likewise, in a rat model of traumatic brain injury (a risk factor for Alzheimer's), rats treated with vitamin E exhibited no increase in Abeta peptides or cognitive dysfunction, in contrast to rats not receiving vitamin E. A preferred daily dosage includes at least about 100 mg of vitamin C as ascorbic acid or dehydroascorbic acid and at least about 200 mg of vitamin E.

L-Carnosine (b-alanyl-L-Histidine) is a naturally occurring di-peptide of the amino acids alanine and histidine. It is found in brain, muscle and other innervated tissues. High concentrations of carnosine are present in long-lived cells such as neuronal tissues and may be an aging marker. Carnosine, a powerful antioxidant, is active against by-products and metabolites caused by reactive oxygen species as well as an anti-glycosylation effect. MDA (malondialdehyde), a marker of DNA damage from oxidative stress is blocked by carnosine.

Carnosine both prevents sugar aldehydes from reacting with the amino acid on protein molecules as well as reversing the process. Carnosine's protection against cross-linking and the formation of abnormal AGEs, and its ability to reduce or prevent cell damage caused by beta amyloid provides anti-aging benefits. In an 8 week study using L-carnosine, children with autistic spectrum disorders showed statistically significant improvements on the Gilliam Autism Rating Scale (total score and the Behavior, Socialization, and Communication subscales) and the Receptive One-Word Picture Vocabulary test (all P<0.05). Improved trends were noted on other outcome measures. Although the mechanism of action of L-carnosine is not well understood, it may enhance neurologic function, perhaps in the enterorhinal or temporal cortex. When included in an AD treating composition L-carnosine is preferably present in daily dosages of at least about 100 mg.

Epigallocatechin-3-gallate (EGCG), a polyphenol commonly recovered from green tea extract, which is standardized to a minimum of 50% EGCG, is a potent anti-inflammatory and antioxidant compound. EGCG is believed to be involved in amyloid precursor protein (APP) secretion and protection against toxicity induced by beta-amyloid. EGCG can reuse PC₁₂ cells against Abeta toxicity. Experimental evidence suggests that green tea can improve age-related cognitive decline and confer neuroprotection in Alzheimer's Disease models. Although initially ascribed to the antioxidant properties of green tea, the neuroprotective effects may be due to a wide spectrum of cellular signaling events targeting many disease processes.

In cultured hippocampal neurons exposed to Abeta for a 48-hour period, co-treatment of the cells with EGCG decreased the levels of malondiadehyde (a marker for glycation) and caspase C (a marker of abnormal platelet function) compared to controls with no EGCG. Cells treated with EGCG also exhibited increased survival compared to controls. Similarly, a water-based extract of green tea inhibited the aggregation of rabbit platelets in vitro. The investigators found that green tea was comparable to aspirin in preventing platelet aggregation. Finally, EGCG was shown to inhibit the inflammatory markers TNF-a and NF-KB, as well as interleukin-1 proinflammatory signal transduction in cultured epithelial cells. It also appears that EGCG may protect against ischemic neuronal damage. EGCG is preferably present in daily dosages of at least about 25 mg.

Complex Vitamins (B-6, B-12, Folate) prevent or reduce homocystein damage. Recent results indicate that elevated homocysteine (HC) levels induce direct neurotoxicity and potentiate ABeta and glutamate neurotoxicity.): Experimental evidence suggests that B vitamins may improve both cognitive functioning and biochemical markers for Alzheimer's Disease processes. In cultured brain cells grown in media deficient in folic acid, the addition of methotrexate (a folic acid inhibitor) to the media rendered nerve cells more susceptible to death from Abeta. Likewise, in a mouse model of Alzheimer's a folic acid-deficient diet resulted in DNA damage and damage to the hippocampus. In patients from the Framingham Heart Study, low levels of plasma B6 were correlated with high levels of the inflammatory marker C-reactive protein. In patients with mild cognitive impairment and increased homocysteine levels, treatment with a B6-B12-folate combination improved blood brain barrier function and appeared to stabilize cognitive status. In in vitro studies, vitamin 131 inhibited formation of advanced glycation end products in bovine serum albumin, ribonuclease A, and human hemoglobin. Low B-12 and Folate blood levels are associated with dementia. Vitamins B-6, Folate and B-12 can reduce these elevated HC levels. Vitamin B-5 (pantothenic acid) is also necessary to form acetylcholine. Additionally, applicant has found that certain lesser known metabolites or alternative forms of some of the B vitamins, such as B-1, B-6 and B-12, play important roles in AD beyond their identified uses for reduction of homocysteine. For example, the hydroxycobalamine form of B-12 has been found to scavenge NO radicals which have been associated with neurodegeneration and migraines. The benfotiamine form of vitamin B-1 has demonstrated significant benefit against excessive glycation and advanced glycation endproducts (AGEs) which have been associated with Abeta, glia inflammation and folate compositions have now been found to address inflammation, for example caused by NO, as well as endothelial function. Nitrogen oxide (NO) synthase creates NO which is inflammatory to tissue. The beneficial properties of folates can also be enhanced by the concurrent use of certain B vitamins, particularly pyridoxal-5-phosphate (P5P) and hydrocobalamin, antioxidants, such as vitamin E, SAMe and CoQ10. Addition of NO synthase inhibitors, such as amino-guanidine, L-carnitine, asymmetric argentine, and certain plant derived phytochemicals can enhance the inflammation reducing properties of folates. A particularly preferred daily dosage comprises 100 mcg. to 10 mg of folate along with one or more of the hydroxycobalamin form of B12 (100 mcg-1 mg), B6 (pyridoxal-5-phosphate or pyridoxamine) (1 mg to 100 mg), and 25 mg-1,000 mg of riboflavin (B2).

Piperine, a component of the spice black pepper, increases the bioavailability of curcurmin and epigallocatechin-3-gallate. Piperine also exhibits significant antioxidant activity of its own, as well as significant chemopreventative and immunomodulary effects. A preferred daily dosage contains at least about 2.5 mg of piperin. A preferred source is piper longum derived from black pepper and standardized as 90%+piperin.

A preferred composition comprises 500 mg of NAC, 100 mg of EGCG from green tea extract, 300 mg of alpha lipoic acid, 5 mg of folic acid, 1,000 mcg of hydroxycobalamin, 50 mg of pyridoxal-5-phosphate (B-6) and/or 50 mg of pyridoxamine (B6), 1,000 mg of turmeric (95% curcumin), 25 mg of vitamin B2, 25 mg of vitamin B1 (benfotiamine), 300 mg of vitamin C and 400 IU of tocopheryl succinate (vitamin E). A preferred composition, set forth by % gross weight is listed in Table 3

TABLE 3
Preferred Medical Food Cocktail

| | % Gross |
|---|---|
| Cocktail Ingredient | Weight |
| Turmeric (standardized to 95% curcurminoids) | 32.1% |
| Piper longum (black pepper standardized to 95% + | 0.7% |
| piperine) | |
| Green tea extract (standardized to 50% Epigallocatechin- | 6.4% |
| 3-gallate | |
| R-α-Lipoic Acid | 9.6% |
| N-Acetylcysteine | 16.0% |
| B1 (Benfotiamine/Thiamine pyrophosphate) | 2.7% |
| B6 (Pyroxidal-5-phosphate/pyridoxamine) | 5.3% |
| B12 (Hydroxycobalamin) | 3.2% |
| Folic Acid/Folate | 1.6% |
| Vitamin C (Ascorbic acid/Dehydroascorbic acid) | 9.6% |
| Vitamin E (Tocopherol succinate) | 12.8% |

The % gross weight for each ingredient in the cocktail was determined by scaling up the elemental or therapeutic levels for each ingredient by its total weight as provided by the raw material suppliers. For example, if the anticipated therapeutic level of EGCG is 100 mg and the green tea extract used is 50% EGCG, then the gross weight of the green tea extract would be 200 mg.

Standardization of the content of all herbal products (tumeric, piper longum, and green tea) was confirmed by certificate of analysis from the supplier and also by assay by an independent laboratory of the herbal products.

Turmeric and green tea were obtained from USA NutraSource (City of Industry Calif.), black pepper was obtained from Sabinsa Corporation (Piscataway, N.J.), benfotiamine (B1), pyridoxamine (B6), and hydroxycobalamin (B12) were obtained from Sigma Aldrich Corporation (St. Louis, Mo.), N-acetylcysteine was obtained from Ashland Chemical (Cleveland, Ohio), α-Lipoic acid, vitamin B12, folic acid, and vitamin E were obtained from Stauber Ingredients (Fullerton, Calif.) and vitamin C in the form of ascorbic acid and dehydroascorbic acid was obtained from Harmony Concepts (Eugene, Oreg.).

Tumeric was used as the main dosing reference. In test studies mice were given a) no cocktail (control group), b) a low dose cocktail (10 μM curcumin) or c) a high dose cocktail (50 μM curcurmin).

While specific formulations or combinations of compounds have been set forth as beneficial or preferred, the invention is not limited to those combinations, or compositions listed herein as other compounds, whether natural, or synthesized may be discovered to be active in treating or preventing neurological and cognitive disorders. The invention is limited only by the claims set forth herein which include folate in combination with other active ingredients for prevention, or treatment or reduction of the symptoms of neurological or cognitive disorders, particularly Alzheimer's Disease.

It is preferred that these compositions be delivered orally and the components be prepared for ingestion in a manner that makes the composition available in therapeutically effective amounts. As such, they may be prepared as water soluble compositions, delivered in liquid form, lyophilized, encapsulated, or in a manner suitable for time release, delayed release or enteric delivery, or any manner typically used for orally delivered pharmaceuticals, nutraceuticals or vitamins, or combined with foods or other normally ingested products. However, the invention is not limited to oral delivery as the compositions set forth herein may also be delivered by nasal spray, inhalation techniques, transdermally, transmucossal, by suppository, injected or by intravenous methods.

1. A medicinal composition comprising compounds effective for reducing or preventing oxidative stress, glycation or glycosylation and inflammation and normalize platelet function and homocysteine levels, said compounds present in quantities effective for preventing, treating or reducing the symptoms of neurological diseases or cognitive deficiencies.

2. The medicinal composition of claim 1 comprising at least three compounds selected from:
a) curcumin,
b) alpha lipoic acid,
c) N-acetycysteine,
d) vitamin C or vitamin E,
e) epigallocatechin-3-gallate,
f) B-complex vitamins selected comprising folate and one or more of vitamin B2, vitamin B6, vitamin B12, or derivatives thereof, and
g) piperin.

3. The medicinal composition of claim 1 comprising a daily dose of at least three compounds selected from:
a) at least about 250 mg of curcumin,
b) at least about 50 mg of alpha lipoic acid,
c) at least about 100 mg of N-acetycysteine,
d) at least about 100 mg of vitamin C in the form of ascorbic acid or dehydroascorbic acid or at least about 200 mg of vitamin E,
e) at least about 25 mg of epigallocatechin-3-gallate,
f) at least about 100 mcg folate and one or more of at least about 25 mg of vitamin B2, at least about 1 mg of vitamin B6 in the form of pyridoxal-5-phosphate or pyridoxamine, at least about 100 mcg of the hydroxycobalamin form of vitamin B 12, or derivatives thereof, and
g) at least about 2.5 mg of piperin.

4. The medicinal composition of claim 1 comprising a daily dose of 500 mg of NAC, 100 mg of EGCG from green tea extract, 300 mg of alpha lipoic acid, 5 mg of folic acid, 1,000 mcg of hydroxycobalamin, 50 mg of pyridoxal-5-phosphate (B-6), 1,000 mg of turmeric (95% curcumin), 25 mg of vitamin B2, 25 mg of vitamin B1 (benfotiamine), 300 mg of vitamin C, 400 IU of tocopheryl succinate (vitamin E) and 5 mg of piperin.
